(19) United States
(12) Patent Application Publication
(10) Pub. No.: US 2026/0314590 A1
(43) Pub. Date: Oct. 15, 2026

(54) Waterfowl Decoy Having Feeding Motion on Water Surface

(71) Applicant: Good Sportsman Marketing, LLC, Irving, TX (US)

(72) Inventors: Damon L. Coalson, Dallas, TX (US); Fred L. Zink, JR., Port Clinton, OH (US)

(21) Appl. No.: 19/640,361

(22) Filed: Apr. 6, 2026

Related U.S. Application Data
(60) Provisional application No. 63/786,150, filed on Apr. 9, 2025.

(51) Int. Cl. A01M 31/06

(52) U.S. Cl. CPC A01M 31/06

(57) ABSTRACT

A waterfowl decoy mimicking feeding on the water surface has a sealed chamber and is buoyant in the water. An actuator disposed in the sealed chamber includes a motor, a screw, and a yoke. The screw is rotatable in response to the motor’s drive to displace the yoke. The decoy has a level orientation at the water surface when the yoke is in one position along the screw, and the decoy has a tilted orientation with the head submerged below the surface and the tail located at the surface when the yoke is in another position along the screw. Circuitry housed in the sealed chamber controls the actuator to displace the yoke and mimic feeding motions. An anchor used below the surface has a tether connected to the decoy. The anchor houses a power supply, and the tether conducts power from the power supply to the circuitry and actuator.

### CROSS-REFERENCE TO RELATED APPLICATIONS

This application claims the benefit of U.S. Provisional Appl. No. 63/786,150 filed Apr. 9, 2025, which is incorporated herein by reference in its entirety.

### BACKGROUND OF THE DISCLOSURE

Decoys for waterfowl can be used to create the illusion of a safe, attractive gathering spot where the waterfowl can land, feed, or rest. For example, ducks are social birds and tend to flock toward other ducks when migrating or looking for safe places to land. When ducks see what looks like a group already on the water, the ducks are more likely to come in range of the hunters, believing it is a safe and active spot.

The use of decoys for waterfowl, such as ducks, is thousands of years old and is one of the oldest hunting tools in North America. Earliest decoys dating back over 2,000 years were made by Native American tribes (like the Paiute) and have been found in caves around present-day Nevada. These decoys used bulrushes and feathers. In the 1800s and early 1900s, hunters made hand-carved, hand-painted wooden decoys, and decoy carving became an American folk art.

Modern decoys today are often made of plastic, foam, or lightweight materials, with detailed paint jobs and sometimes built-in movement features. Traditionally, decoys are buoyant devices that merely float on the water surface and may be anchored in place using anchors. Hunters strategically place multiple decoys (called a spread) on the water in realistic patterns to mimic the behavior of live ducks. Hunters use patterns, numbers, and positioning in the spread based on duck behavior, weather, and location. The decoys can be stationary or motion-based (like spinning wing decoys) to add movement and make the setup more convincing.

Hunters may use species-specific decoys (mallards, pintails, teal) to match local flocks. Early in the season, hunters may use smaller spreads but may use much larger spreads late in the season when ducks are flocking heavily. Different spread patterns can be used. In J-Hook or Fishhook patterns, for example, ducks tend to land at the open end of the hook. In U-Shape or Horseshoe patterns, ducks naturally land in the opening. Ducks prefer to land into the wind so hunters will often leave an open pocket upwind, steering the ducks toward that area. Additionally, the decoys are usually spaced with enough room between them to look realistic.

Ducks are social and cautious. If the ducks see decoys that sit stiff in flat, still water, the ducks get suspicious. But if there are natural-looking movements, splashes, and ripples, the ducks are much more likely to commit and land. To make the decoys more lifelike to attract waterfowl, various techniques have been used.

In one of the oldest and simplest techniques, a jerk string is attached to a few decoys and anchored with a weight. The hunter sits in the blind holding the end of the line. When hunter tugs the string, the decoys bob, ripple the water, and mimic the subtle movement of feeding ducks.

To make the decoys more lifelike to attract waterfowl, various motorized mechanisms have been added to the decoys to mimic flapping wings, feeding movements, and the like. For example, hunters may use motion decoys that include spinning wings or swimming decoys to add natural movement, especially on calm days when the water is still. Some brief examples are disclosed in US5377439, US6088944, US6412209, US2004/0010957, US2006/0207158, US2014/0259860, US2017/0049095, US11083189, and US2023/0380411.

For example, in one mechanized technique, a pulsator or bubbling decoy is battery-powered and has a small pump inside. The decoy sits low in the water and periodically pulse water jets underneath, creating ripples and movement.

A feeder butt decoy is shaped like an upside-down duck, showing just the tail and rear end sticking up—exactly like a duck tipping forward to feed. Hunters often include a few of these to visually signal to other ducks that the water is a safe, food-rich spot.

A swimmer decoy actually floats in small circles or moves forward slowly via a motor or cord. This imitates the subtle back-and-forth or slow drifting movement ducks make while nibbling around the water’s surface.

To make ripples, separate devices (e.g., floating battery-powered pucks) can be used to create gentle ripples and movement across the whole spread. Even stationary decoys look more lifelike when the water surface is not perfectly still. Finally, a wind-driven decoy can have small paddles underneath that spin when the wind blows, causing the decoy to bob, rock, or splash lightly.

Although the above techniques may be effective, hunters are always striving to making decoy look as “alive” and natural as possible.

The subject matter of the present disclosure is directed to overcoming, or at least reducing the effects of, one or more of the problems set forth above.

### SUMMARY OF THE DISCLOSURE

In one configuration, an assembly disclosed herein is used to mimic a waterfowl feeding on a water surface. The assembly comprises a decoy, an actuator, and circuitry. The decoy has first and second ends and has a sealed chamber. The decoy is buoyant on the water surface. The actuator is disposed in the sealed chamber and comprises a motor, a screw, and a yoke. The motor is operable to produce a drive, which rotates the screw. The yoke is disposed on the screw and is displaceable on the screw between at least first and second positions in response to the rotation. The decoy has a first orientation at the water surface in response to the yoke in the first position, while the decoy has a second orientation at the water surface in response to the yoke in the second position. The circuitry in electrical communication with the actuator is configured to operate the motor to displace the yoke between the first and second positions.

In another configuration, an assembly disclosed herein is used to mimic a waterfowl feeding on a water surface. The assembly comprises a decoy, a weight, an actuator, and circuitry. The decoy, which is buoyant on the water surface, has first and second ends and has a sealed chamber. The weight is disposed in the sealed chamber, and the actuator is disposed in the sealed chamber and is actuatable to shift the weight between first and second positions. The weight in the first position permits the first end to be buoyant on the water surface, and the weight in the second position tilts the first end below the water surface. The circuitry in electrical communication with the actuator is configured to shift the weight between the first and second positions.

In yet another configuration, an assembly disclosed herein is for use on a water surface. The assembly comprises a decoy, an actuator, circuitry, and an anchor. The decoy, which is buoyant on the water surface, has first and second ends and has a sealed chamber. The actuator is disposed on the sealed chamber and comprises a motor, a screw, and a yoke. The motor is operable to produce a drive, and the screw is rotatable with a rotation in response to the drive of the motor. The yoke is disposed on the screw and is displaceable on the screw between at least first and second positions in response to the rotation. The decoy has a level orientation with the first and second ends at the water surface in response to the yoke in the first position. Meanwhile, the decoy has a tilted orientation with the first end submerged below the water surface and the second end of the decoy located at the water surface in response to the yoke in the second position. The circuitry is housed in the sealed chamber. The circuitry, which is in electrical communication with the actuator, is configured to operate the motor to displace the yoke between the first and second positions. The anchor is useable below the water surface and has a tether connected to the decoy at the water surface. The anchor houses a power supply, and the tether has conductors conducting power from the power supply to the circuitry.

In various configurations, a bearing can be disposed in the sealed chamber to support a distal end of the screw. The sealed chamber can house at least a portion of the circuitry. Additionally, the decoy can comprise a buoyant chamber disposed toward the second end. The buoyant chamber can be configured to provide buoyancy to the decoy.

The decoy in the first orientation can have a level orientation with the first and second ends at the water surface in response to the yoke in the first position. Meanwhile, the decoy in the second orientation can have a tilted orientation with the first end submerged below the water surface and the second end of the decoy located at the water surface in response to the yoke in the second position.

The decoy can define an opening toward the first end. The opening can be configured to take in water when the decoy is changed from the level orientation to the tilted orientation. The opening can be configured to let out water when the decoy is changed from the tilted orientation to the level orientation.

When the assembly comprises an anchor useable below the water surface, the anchor can house at least a portion of the circuitry, and the tether can have conductors conducting control to the actuator. Additionally, or alternatively, the anchor can house a power supply, and the tether can have conductors conducting power from the power supply to the actuator.

A reducer can be connected between the motor and the screw and can reduce shaft rotation of the motor to the drive for the screw. Further, the screw can comprise a worm gear feed screw, and the yoke can comprise a nut disposed on the worm gear feed screw. Alternatively, the screw can be a rotatable screw of a ball screw assembly, and the yoke can comprise a ball nut of the ball screw assembly disposed on the rotatable screw. The yoke can have a weight associated therewith.

The circuitry can comprise a timer at least having a first time interval for the decoy having the first orientation and a second time interval for the decoy having the second orientation. The circuitry can comprise at least one contact switch disposed in the sealed chamber. The at least one contact switch detects the yoke in at least one of the first and second positions on the screw.

The decoy can comprise at least one foot hingedly connected thereto. The actuator can comprise a second motor and a converter. The second motor can be operable to produce rotation. The converter can be connected between the second motor and the at least one foot. The converter can be configured to convert the rotation of the second motor into flapping of the at least one foot.

In any of the configurations, the assembly can further comprise at least one foot hingedly connected to the decoy. The actuator can be actuatable to reciprocate the at least one foot. The actuator can comprise a motor and a screw. The motor can be operable to produce a drive, and the screw can be rotatable with a rotation in response to the drive of the motor. The weight can be displaceable between at least the first and second positions in response to the rotation or the screw.

The foregoing summary is not intended to summarize each potential embodiment or every aspect of the present disclosure.

### BRIEF DESCRIPTION OF DRAWINGS

FIG. 1A illustrates a side elevational view of a waterfowl decoy assembly according to the present disclosure.

FIG. 1B illustrates a front elevational view of the waterfowl decoy assembly.

FIG. 1C illustrates a perspective view of the waterfowl decoy assembly.

FIG. 2 illustrates a perspective view of an anchor for the waterfowl decoy assembly.

FIG. 3A illustrates a front view of the waterfowl decoy assembly.

FIG. 3B illustrates a cross-sectional view of the waterfowl decoy assembly.

FIGS. 4A-4B illustrate cross-sectional views of the waterfowl decoy assembly in states of operation.

FIG. 5 illustrates a cross-sectional view of another waterfowl decoy assembly of the present disclosure having a power supply on the decoy.

FIG. 6 illustrates a cross-sectional view of yet another waterfowl decoy assembly of the present disclosure having movable feet on the decoy.

FIG. 7 schematically illustrates control circuitry for the disclosed waterfowl decoy assembly.

### DETAILED DESCRIPTION OF THE DISCLOSURE

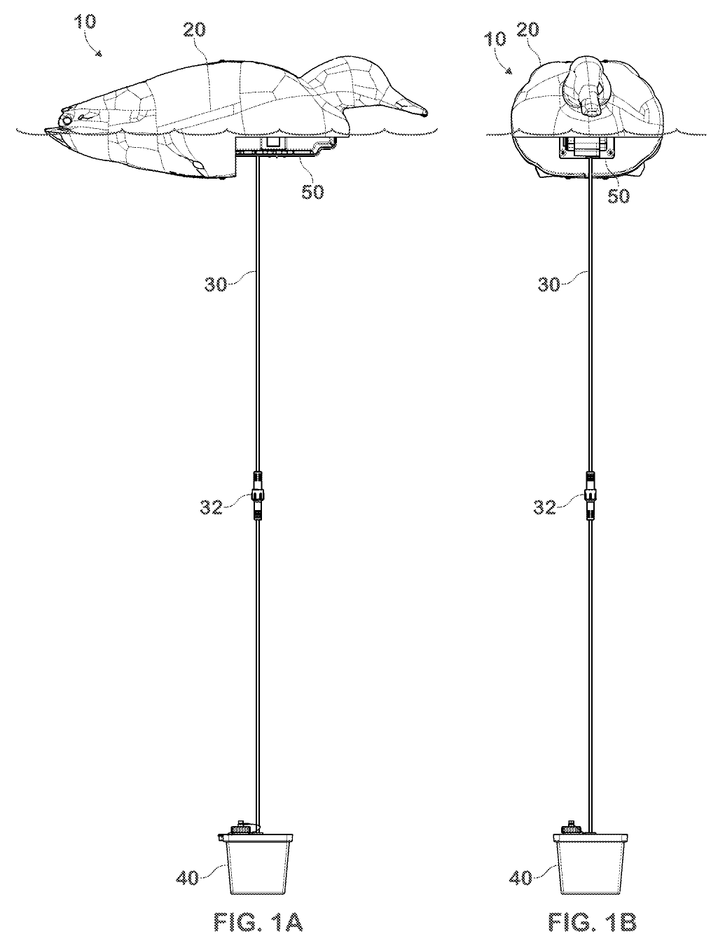

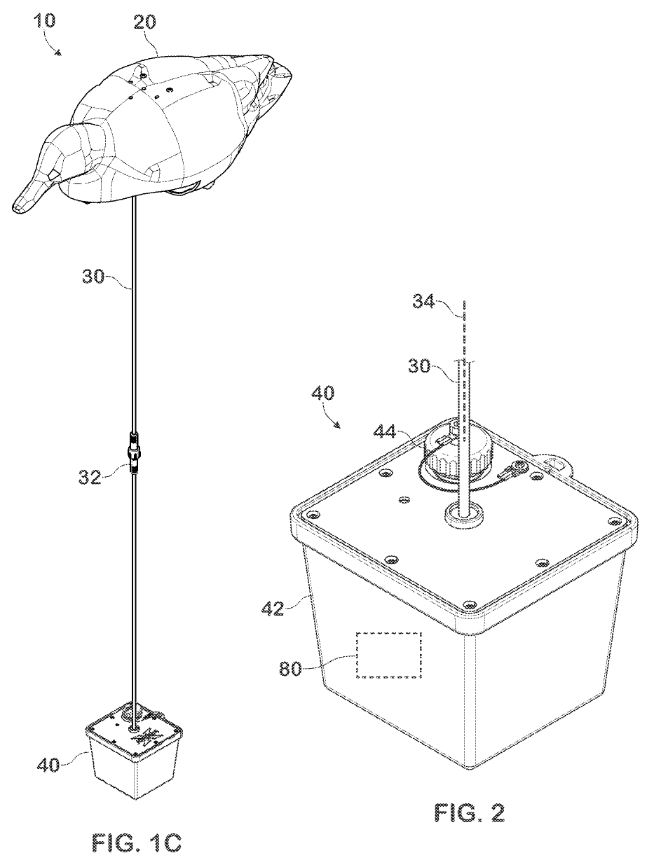

FIGS. 1A-1B illustrate side and front elevational views of a waterfowl decoy assembly 10 according to the present disclosure. FIG. 1C illustrates a perspective view of the waterfowl decoy assembly 10. The waterfowl decoy assembly 10 includes a decoy 20, an actuator 50, a tether 30, and an anchor 40. The decoy or body 20 is buoyant on the water surface. The anchor 40 is used below the water surface and connects by the tether 30 to the decoy 20, which floats at the water surface.

The decoy 20 is designed to mimic a waterfowl, floating on the water surface, tipping down below the water surface to feed below the surface, and then tipping back up to the water surface. For example, the decoy assembly 10 is operated to emulate a duck on the water surface that can dip to emulated feeding under water. The decoy 20 is tethered to the anchor 40, which can also act as a power supply for the actuator 50. For example, battery power in the anchor 40 can power the actuator 50 on the floating decoy 20 at the water surface. As discussed in more detail below, the actuator 50 has an electric motor, a reducer (e.g., gear reducer), a drive screw, and a weighted yoke to create a weight transfer to make the decoy 20 tip up/down and create movement and wake. The design is all self-contained with no visually moving parts, and the movement provided by the actuator 50 is intended to be realistic. In a further configuration, movable feet can be included on the decoy 20.

FIG. 2 illustrates a perspective view of the anchor 40 for the waterfowl decoy assembly 10. The anchor 40 includes a weighted container 42. As noted above, the weighted container 42 can house a power supply 80, such as one or more batteries. The weighted container 42 is waterproof and may have a sealed cap 44 for internal access. Conductors 34 can be used in the tether 30 to connect the power supply 80 to the actuator 50 on the decoy 20 at the water surface. At least some of the control circuitry for the actuator 50 may also be contained in the weighted container 42 and connected by conductors 34. As shown in FIGS. 1A-1C, a connector 32 can be used on the tether 30 to connect the decoy 20 to the anchor 40.

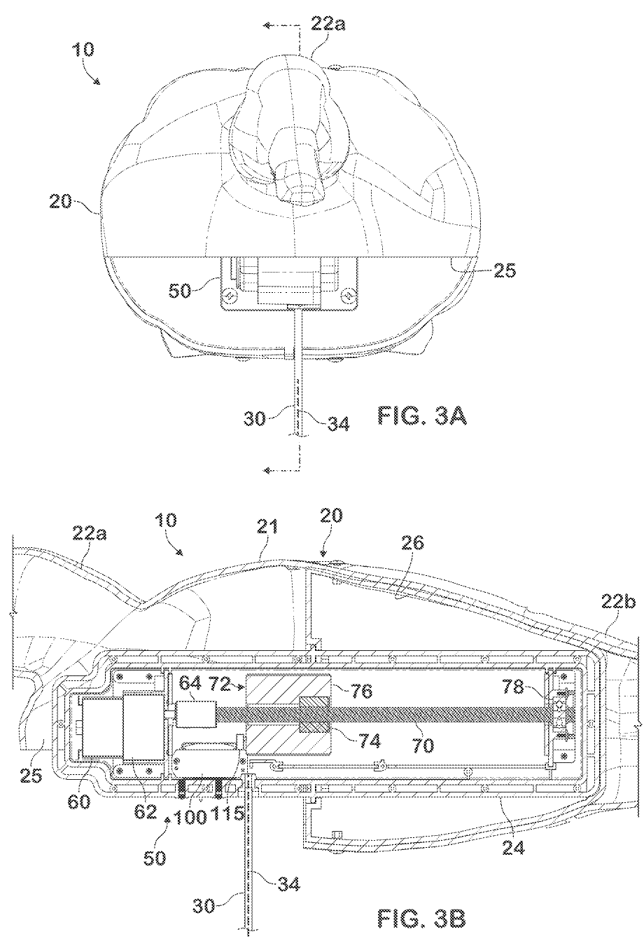

Turning to more details, FIG. 3A illustrates a front view of the waterfowl decoy assembly 10, and FIG. 3B illustrates a cross-sectional view of the waterfowl decoy assembly 10. As noted above, the waterfowl decoy assembly 10 includes the decoy 20, the actuator 50, and the tether 30, which connects to the anchor (40). The decoy or body 20 has a first end or head 22a and has a second end or tail 22b. The decoy 20 has a shell 21 and a sealed chamber 24. The shell 21 can be shaped and colored to mimic a waterfowl, such as a duck. In addition to the seal chamber 24 for the actuator 50, the decoy 20 can include a buoyant chamber 26 disposed toward the second end 22b and being configured to provide buoyancy to the decoy 20.

The actuator 50 is disposed on the sealed chamber 24 and includes a motor 60, a drive screw 70, and a yoke 72. The motor 60 can be an electric motor having low revolutions per minute. The motor 60 is operable to produce a drive, and the drive screw 70 is rotatable with a rotation in response to the drive of the motor 60. The yoke 72 is disposed on the screw 70 and is displaceable along the screw 70 between at least first and second positions in response to the rotation. As discussed in more detail below, when the yoke 72 is in a first (backward) position, the decoy 20 has a first (level) orientation in the water (i.e., with the first and second ends 22a-b at the water surface). When the yoke 72 is in the second (forward) position, however, the decoy 20 has a second (tilted) orientation in the water (i.e., with the first end 22a submerged below the water surface and the second end 22b of the decoy 20 located at the water surface).

The decoy 20 can define an opening 25 toward the first end 22a. The opening 25 can take in water when the decoy 20 is changed from the level orientation to the tilted orientation in the water. Also, the opening 25 can let out water when the decoy 20 is changed from the tilted orientation back to the level orientation.

Looking at the actuator 50 in more detail, at least a portion of the control circuitry 100 can be housed in the sealed chamber 24 of the decoy 20 and can receive power from the tether’s conductors 34 connected to the power supply (80) in the anchor (40). The control circuitry 100 is in electrical communication with the motor 60 and is configured to operate the motor 60 to displace the yoke 72 between the first and second positions.

Although the power supply (80) can be housed in the anchor (40), other configurations can be used if there is enough available space. For example, the weight associated with the yoke 72 can incorporate at least part of the power supply (80), such as a battery, for the control circuitry 100 and the motor 60. Because such a power supply (80) would move with the yoke 72, appropriate electrical connections can be used.

A bearing 78 is disposed in the sealed chamber 24 and supports a distal end of the screw 70. The motor 60 connects to a reducer 62, which connects to the screw 70 with a coupling 64. The reducer 62 reduces shaft rotation of the motor 60 to the drive for the screw 70. Meanwhile, the screw 70 can be a worm gear feed screw, and the yoke 72 can have a nut 74 disposed on the worm gear feed screw 70. Likewise, the screw 70 can be a rotatable screw of a ball screw assembly, and the yoke 72 can have a ball nut 74 of the ball screw assembly disposed on the rotatable screw 70. Based on the weight of the decoy 20 and its buoyancy, the yoke 72 can have a defined weight 76 associated with it.

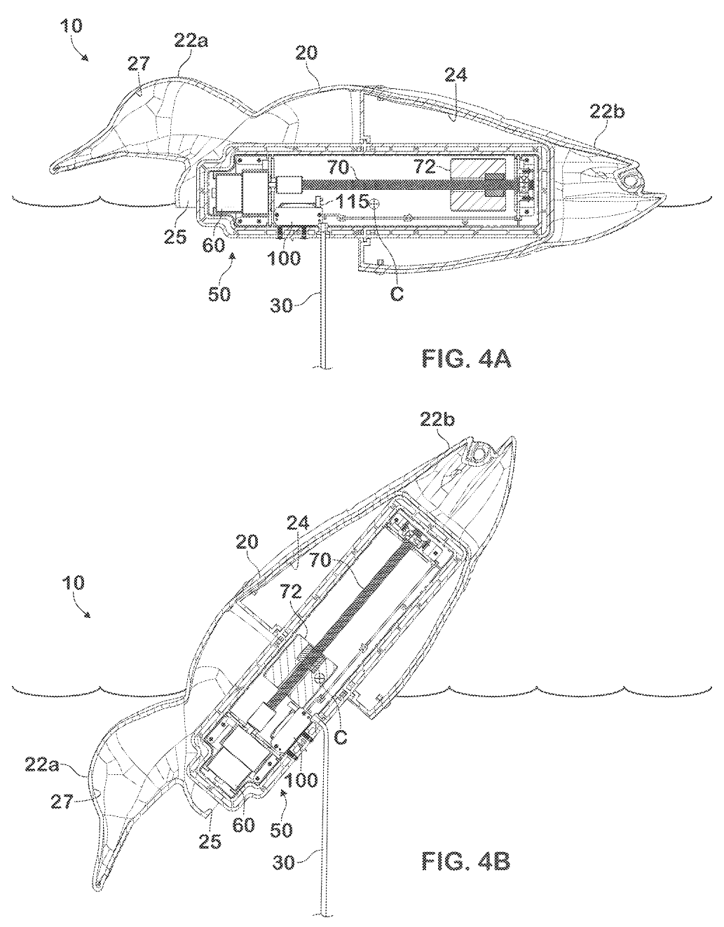

Having an understanding of the assembly 10, discussion turns to the operation of the assembly 10. FIGS. 4A-4B illustrate cross-sectional views of the waterfowl decoy assembly 10 in states of operation. As noted above, the actuator 50 can move (tilt, dip, tip, etc.) the decoy 20 between first and second orientations at the water surface based on the movement and position of the yoke 72 on the screw 70. As shown in FIG. 4A, the decoy 20 in the first orientation has a level orientation with the first and second ends 22a-b located at the water surface in response to the yoke 72 in the first (backward) position. Meanwhile, the decoy 20 in the second orientation shown in FIG. 4B has a tilted orientation with the first end 22a submerged below the water surface and the second end 22b of the decoy 20 located at (and optionally above) the water surface in response to the yoke 72 in the second (forward) position.

When the motor 60 is turned in one direction, for example, the drive rotates the screw 70 so that the yoke 72 travels from back to front. As the yoke 72 comes closer to the motor 60, it passes a center of buoyancy (C) of the decoy 20, causing the decoy 20 to tip, tilt, or dip forward. (The center of buoyancy (C) is only schematically illustrated in FIGS. 4A-4B.) For example, the head 22a can dip down, and the tail 22b can lift up during this tilting. When the motor 60 is turned in a reverse direction, the drive rotates the screw 70 so that the yoke 72 travels from front to back. As the yoke 72 moves further from the motor 60, it passes the center of buoyancy of the decoy 20 and balances with the weight of the motor 60, reducer 62, etc., causing the decoy 20 to float level on the water surface.

Again, as noted, the decoy 20 can define the opening 25 toward the head 22a. The opening 25, which does not communicate with the sealed chamber 24 nor the buoyant chamber 26, can take in water when the decoy 20 is changed from the level orientation (FIG. 4A) to the tilted orientation (FIG. 4B) in the water. Also, the opening 25 can let out water when the decoy 20 is changed from the tilted orientation (FIG. 4B) back to the level orientation (FIG. 4A). In fact, the head of the decoy 20 can form an internal pocket 27 to fill with water to help keep the first end submerged, but to empty of water when the decoy 20 is brought level at the water surface.

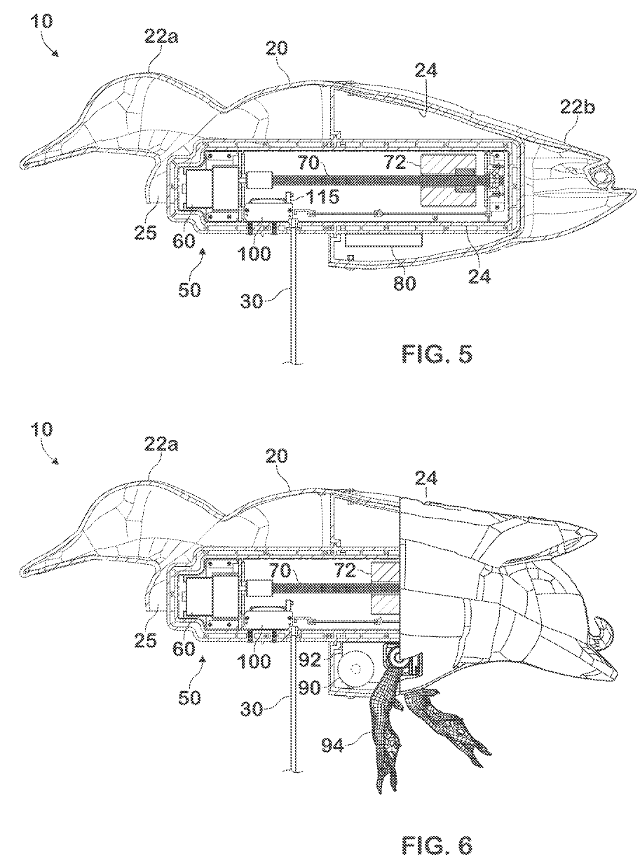

FIG. 5 illustrates a cross-sectional view of another waterfowl decoy assembly 10 of the present disclosure. Should weight and power requirements permit, the power supply 80 can be disposed on the decoy 20, being housed in the buoyant chamber 26 or in the sealed chamber 24 for the actuator 50. The tether 30 may still connect to an anchor, but the tether 30 may not require conductors for delivering power. The placement of the power supply 80 can be configured relative to the weighted yoke 72 so that the buoyancy of the decoy 20 can still be exploited to dip the decoy 20 as noted herein.

FIG. 6 illustrates a cross-sectional view of yet another waterfowl decoy assembly 10 of the present disclosure. Should weight and power requirements permit, the decoy 20 can have movable feet 94 hingedly connected to the decoy 20. The tilting or tipping motor 60 of the actuator 50 could be configured to move the movable feet 94 given proper gearing and linkages. More simply, the actuator 50 can use a second motor 90 housed in the buoyant chamber 26 or in the sealed chamber 24 for the actuator 50. In general, the second motor 90 can provide rotation and can use a mechanical converter 92 converting the rotation of the second motor 90 to flapping/paddling motion for the feet 94. Other than a second electric motor 90, the actuator 50 can use other types of actuating devices, such as a linear actuator, a solenoid, or the like, to produce the paddling motion. The placement of the motor 90 and the mechanical converter 92 can be configured relative to the weighted yoke 72 so that the buoyancy of the decoy 20 can still be exploited to dip the decoy 20 as noted herein.

The mechanical converter 92 can convert rotation of the motor 90 to flapping motion of the feet 94. When the decoy 20 is tipped to mimic feeding, for example, the feet 94 can be paddled, as would typically be done by a duck to keep balance when tipping. The mechanical converter 92 can use any number of mechanisms (e.g., reciprocating linkage, cam and follower, scotch yoke, gear and linkage, etc.) to convert motor rotation to feet flapping. Details of mechanisms for the mechanical converter 92 are disclosed in co-pending Application No. 19/640,346 filed concurrently herewith and incorporated herein by reference.

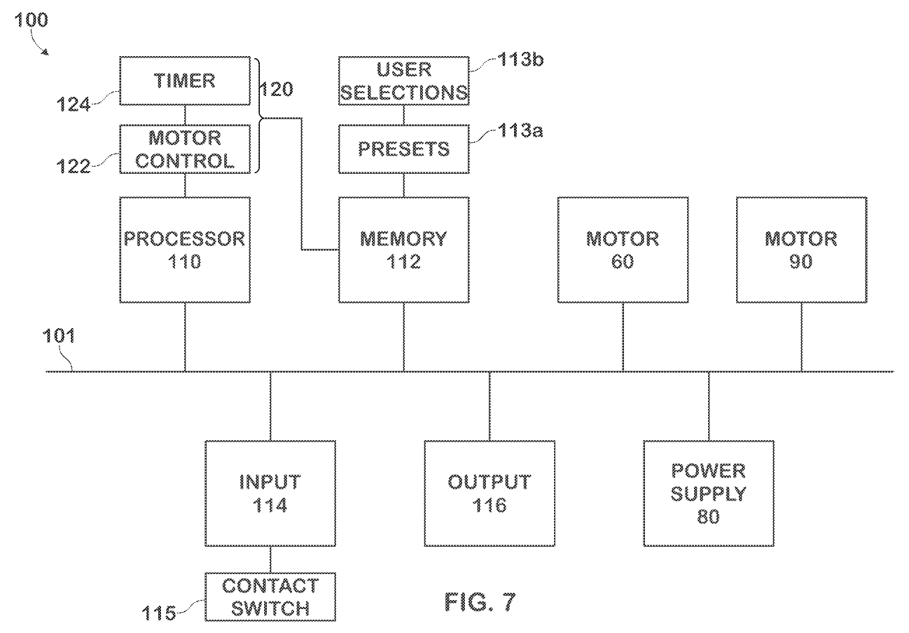

FIG. 7 schematically illustrates control circuitry 100 for the disclosed waterfowl decoy assembly 10. As already mentioned, the control circuitry 100 can be housed in the anchor 40 or in the decoy 20, or portions of the control circuitry 100 can be housed in both. The control circuitry 100 can include a bus 101, a processor 110, a memory 112, and interface(s), such as an input component 114 and an output component 116.

The bus 101 includes one or more components that enable wired and/or wireless communication among the components of the control circuitry 100. The bus 101 can couple together two or more components of the control circuitry 100, motor 60, and power supply 80 via operative coupling, communicative coupling, electronic coupling, and/or electric coupling. The processor 110 can include one or more of a microprocessor, a microcontroller, a field-programmable gate array, an application-specific integrated circuit, and/or another type of processing component. The processor 110 is implemented in hardware, firmware, or a combination of hardware and software.

The memory 112 includes volatile and/or nonvolatile memory. For example, the memory 112 can include random access memory (RAM), read only memory (ROM), a flash memory, and/or another type of memory. The memory 112 stores information, instructions, and/or software 120 related to the operation of the control circuitry 100. The software 120 can include a motor control module 122 to control the motor 60 and can include a timer module 124 to time the operation of the decoy assembly (10). The input component 114 enables the control circuitry 100 to receive input, such as user input and/or sensed input. The output component 116 enables the control circuitry 100 to provide output, such as via a display, a speaker, and/or a light-emitting diode.

For example, the input component 114 can include a button, a switch, a sensor, an accelerometer, a gyroscope, and/or an actuator. Buttons and switches can be used to turn the control circuitry 100 on and off and to set user selections for operation. Sensors can detect the orientation of the decoy (20) during operation. As one example, the control circuitry 100 can include at least one contact switch 115 disposed in the sealed chamber 24. The at least one contact switch 115 to detect the yoke 72 in at least one of the first and second positions on the screw 70. (For instance, FIG. 4B schematically shows a contact switch 115 in the sealed chamber 24 that can be contacted by the yoke 12 when moved forward toward the motor 60.) These and other forms of buttons, switches, and sensors can be used for the input components 114.

The control circuitry 100 can perform one or more operations or processes described herein. For example, the memory 112 can store a set of instructions (e.g., one or more instructions or code) in the software 120 for execution by the processor 110. The processor 110 can execute the set of instructions to perform one or more operations or processes described herein. In some implementations, execution of the set of instructions, by the processor 110, causes the processor 110 and/or the control circuitry 100 to perform one or more operations or processes described herein. In some implementations, hardwired circuitry can be used instead of or in combination with the instructions to perform one or more operations or processes described herein. Additionally, or alternatively, the processor 110 can be configured to perform one or more operations or processes described herein. Thus, implementations described herein are not limited to any specific combination of hardware circuitry and software.

For instance, the memory 112 can store presets 113a and user selections 113b that control the operation of the control circuitry 100, such as defining time intervals of the timer 124 used by the motor control module 122 in controlling the motor 60. For instance, the timer 124 can have a first time interval that defines how long the decoy 20 maintains the level orientation (FIG. 4A) in the water, and the timer 124 can have a second time interval that defines how long the decoy 20 maintains the tilted orientation (FIG. 4A) in the water. Additional time intervals may define speeds of the motor 60 in transitioning between the orientations to define how quickly the decoy transitions from the level orientation (FIG. 4A) to the tilted orientation (FIG. 4A) in the water and de-transitions from the tilted orientation (FIG. 4A) to the level orientation (FIG. 4A). Additional time intervals may also introduce intermediate stoppage of the motor 60 during the transitions. The timers 124 can have presets 113a and can be set by user selections 113b if desirable. The goal of the timers 124 is to make the motion of the decoy 20 more realistic and lifelike to mimic the natural motions of a duck or other waterfowl in the water.

In general, the software 120 is designed to turn the actuator 50 (motor(s) 60, 90) on and off. For instance, the tilting or tipping motor 60 of the actuator (50) is reversable to move the weighted yoke (72) to track forward and rearward between two positions along the screw (70). The software 120 communicates to the motor 60 to power the motor on and to turn the screw (70) and shift the weighted yoke (72). When the weighted yoke (72) is moved towards the head (22a) of the decoy (20), the resulting transfer of weight forward on the decoy 20 changes the buoyant balance of the decoy (20) so that the decoy (20) tips with the head (22a) tipping down and the tail (22b) tipping up. This motion emulates a duck feeding. Once the weighted yoke (72) is all the way forward, the weighted yoke (72) can contact a contact switch (115). In response, the software 120 can then pause for a short time interval and can subsequently actuate the motor 60 to rotate in reverse. This returns the weighted yoke (72) rearward to return the decoy (20) to a level orientation to float naturally of the water surface. A number of other sensing techniques, such an inclinometer, mercury switch, etc., can be used to detect when the decoy (20) is tilted and/or level.

When feeding on the water surface, ducks (especially dabbling ducks like mallards, pintails, or teal) make some distinct motions. The control circuitry 100 can control the motor 60 to move the weighted yoke (72) to mimic one or more of the distinct motions. To mimic tipping or “dabbling,” for example, the control circuitry 100 can tip the decoy (20) forward, dipping the head (22a) underwater while the tail (22b) sticks up in the air. If an additional motor 90 is provided, the control circuitry 100 can paddle feet (94) on the decoy (20), as would typically be done by ducks to keep balance when tipping.

To mimic head dipping and nibbling, the control circuitry 100 can tip the head (22a) of the decoy (20) forward and can oscillate the motor 60 to move the weighted yoke (72) back and forth in the forward position to mimic dabbling and pecking at vegetation, insects, or small critters floating on or just under the water. Pecking can also be mimicked by a vibrating motor (not shown) housed in the sealed chamber 24 of the decoy 20 and controlled by the control circuitry 100. Between feeding dips, the control circuitry 100 can oscillate the motor 60 to move the weighted yoke (72) back and forth to mimic quick bobs between feeding dips. While feeding, ducks might paddle and splash the water. To mimic this to at least some extent, the tipping of the decoy (20) by the control circuitry 100 is intended to produce a wake in the water behind the decoy (20).

The foregoing description of preferred and other embodiments is not intended to limit or restrict the scope or applicability of the inventive concepts conceived of by the Applicants. It will be appreciated with the benefit of the present disclosure that features described above in accordance with any embodiment or aspect of the disclosed subject matter can be utilized, either alone or in combination, with any other described feature, in any other embodiment or aspect of the disclosed subject matter.

In exchange for disclosing the inventive concepts contained herein, the Applicants desire all patent rights afforded by the appended claims. Therefore, it is intended that the appended claims include all modifications and alterations to the full extent that they come within the scope of the following claims or the equivalents thereof.

1. An assembly to mimic a waterfowl feeding on a water surface, the assembly comprising:
a decoy having first and second ends and having a sealed chamber, the decoy being buoyant on the water surface;
an actuator disposed in the sealed chamber and comprising:
a motor being operable to produce a drive;
a screw being rotatable with a rotation in response to the drive of the motor; and
a yoke disposed on the screw and being displaceable on the screw between at least first and second positions in response to the rotation, the decoy having a first orientation at the water surface in response to the yoke in the first position, the decoy having a second orientation at the water surface in response to the yoke in the second position; and
circuitry in electrical communication with the actuator and being configured to operate the motor to displace the yoke between the first and second positions.

2. The assembly of claim 1, comprising a bearing disposed in the sealed chamber and supporting a distal end of the screw.

3. The assembly of claim 1, wherein the sealed chamber houses at least a portion of the circuitry.

4. The assembly of claim 1, wherein the decoy comprises a buoyant chamber disposed toward the second end, the buoyant chamber being configured to provide buoyancy to the decoy.

5. The assembly of claim 1, wherein the decoy in the first orientation has a level orientation with the first and second ends at the water surface in response to the yoke in the first position; and wherein the decoy in the second orientation has a tilted orientation with the first end submerged below the water surface and the second end of the decoy located at the water surface in response to the yoke in the second position.

6. The assembly of claim 5, wherein the decoy defines an opening toward the first end, the opening being configured to take in water when the decoy is changed from the level orientation to the tilted orientation and being configured to let out water when the decoy is changed from the tilted orientation to the level orientation.

7. The assembly of claim 1, further comprising an anchor useable below the water surface and having a tether connected to the decoy at the water surface.

8. The assembly of claim 7, wherein the anchor houses at least a portion of the circuitry, the tether having conductors conducting control to the actuator.

9. The assembly of claim 7, wherein the anchor houses a power supply, the tether having conductors conducting power from the power supply to the actuator.

10. The assembly of claim 1, comprising a reducer connected between the motor and the screw and reducing shaft rotation of the motor to the drive for the screw.

11. The assembly of claim 1, wherein:
the screw comprises a worm gear feed screw, and the yoke comprises a nut disposed on the worm gear feed screw; or
the screw is a rotatable screw of a ball screw assembly, and the yoke comprises a ball nut of the ball screw assembly disposed on the rotatable screw.

12. The assembly of claim 1, wherein the yoke has a weight associated therewith.

13. The assembly of claim 1, wherein the circuitry comprises a timer at least having a first time interval for the decoy having the first orientation and a second time interval for the decoy having the second orientation.

14. The assembly of claim 1, wherein the circuitry comprises at least one contact switch disposed in the sealed chamber, the at least one contact switch detecting the yoke in at least one of the first and second positions on the screw.

15. The assembly of claim 1, wherein the decoy comprises at least one foot hingedly connected thereto; and wherein the actuator comprises:
a second motor being operable to produce rotation; and
a converter being connected between the second motor and the at least one foot and being configured to convert the rotation of the second motor into flapping of the at least one foot.

16. An assembly to mimic a waterfowl feeding on a water surface, the assembly comprising:
a decoy having first and second ends and having a sealed chamber, the decoy being buoyant on the water surface;
a weight disposed in the sealed chamber;
an actuator disposed in the sealed chamber and being actuatable to shift the weight between first and second positions, the weight in the first position permitting the first end to locate at the water surface, the weight in the second position tilting the first end below the water surface; and
circuitry in electrical communication with the actuator and being configured to shift the weight between the first and second positions.

17. The assembly of claim 16, further comprising at least one foot hingedly connected to the decoy, wherein the actuator is actuatable to reciprocate the at least one foot.

18. The assembly of claim 16, wherein the actuator comprises:
a motor being operable to produce a drive; and
a screw being rotatable with a rotation in response to the drive of the motor, the weight being displaceable between at least the first and second positions in response to the rotation or the screw.

19. The assembly of claim 16, wherein the decoy has a level orientation at the water surface in response to the weight in the first position; and wherein the decoy has a tilted orientation in response to the weight in the second position.

20. An assembly for use on a water surface, the assembly comprising:
a decoy having first and second ends and having a sealed chamber, the decoy being buoyant on the water surface;
an actuator disposed on the sealed chamber and comprising:
a motor being operable to produce a drive;
a screw being rotatable with a rotation in response to the drive of the motor;
a yoke disposed on the screw and being displaceable on the screw between at least first and second positions in response to the rotation, the decoy having a level orientation with the first and second ends at the water surface in response to the yoke in the first position, the decoy having a tilted orientation with the first end submerged below the water surface and the second end of the decoy located at the water surface in response to the yoke in the second position; and
circuitry housed in the sealed chamber, the circuitry in electrical communication with the actuator and being configured to operate the motor to displace the yoke between the first and second positions; and
an anchor useable below the water surface and having a tether connected to the decoy at the water surface, the anchor housing a power supply, the tether having conductors conducting power from the power supply to the circuitry.
